(19) United States
(12) Patent Application Publication
(10) Pub. No.: US 2024/0238139 A1
(43) Pub. Date: Jul. 18, 2024

(54) CRANIAL AUTOPSY PROTECTIVE DEVICE FOR THE AUTOPSY OF INFECTED CORPSES

(71) Applicant: Isabella AQUILA, Crotone (IT)

(72) Inventor: Isabella AQUILA, Crotone (IT)

(21) Appl. No.: 18/559,365

(22) PCT Filed: May 6, 2022
(86) PCT No.: PCT/IB2022/054198

§ 371 Date: Nov. 7, 2023

(30) Foreign Application Priority Data
May 7, 2021 (IT) 102021000011822

(51) Int. Cl. A61G 10/00; A61B 90/40; A61G 10/02

(52) U.S. Cl. CPC A61G 10/005; A61B 90/40; A61G 10/02

(57) ABSTRACT

A cranial autopsy protective device for the autopsy of infected corpse, including an aspirating case configured to be positioned on the head of a corpse before the execution of the autopsy allowing the total isolation of the head, the aspirating case including adjustable steel hooks connectable to an autopsy table for the correct adhesion of the aspiration case to the autopsy table. The aspirating case includes a valve with washable rubber insulation through which a nebulization of ozone and chlorine suitable for sanitize the aspirating case is performed after the execution of a craniotomy.

### FIELD

The present invention relates to a cranial autopsy protective device for the autopsy of infected corpses.

In particular, the present invention relates to a cranial autopsy protective device for the autopsy of infected corpses, of the type usable in an autopsy chamber to avoid transmitting infections.

### BACKGROUND

As is known, the autopsy field provides the most important data for understanding the pathogenetic mechanisms of a viral agent, in particular with reference to the Sars COV-2 virus. In fact, in the autopsy field the infectious risk in positive cases is really high, as demonstrated by scientific studies on the permanence of the Sars-COV 2 virus in the cadaver (Analysis of the persistence time of the SARS-COV-2 virus in the cadaver and the risk of passing infection to autopsy staff. Aquila I, Ricci P, Bonetta C F, Sacco M A, Longhini F, Torti C, Mazzitelli M, Garofalo E, Bruni A, Trecarichi E M, Serapide F, Gratteri S, Quirino A, Barreca G S, Abenavoli L, Arena V, Oliva A, Giancotti A, Iavicoli I, Liberto M C, Matera G. Med Leg J. 2021 March; 89 (1): 40-53. Doi: 10.1177/0025817220980601; Postmortem Swabs in the Severe Acute Respiratory Syndrome Coronavirus 2 Pandemic: Report on 12 Complete Clinical Autopsy Cases Dell'Aquila M, Cattani P, Fantoni M, Marchetti S, Aquila I, Stigliano E, Carbone A, Oliva A, Arena V. Arch Pathol Lab Med. 2020 Nov. 1; 144 (11): 1298-1302.)

Up to date, the international autopsy exam includes minimally invasive autopsies to minimize the risk for the operator who is obliged to wear personal protective equipment. Despite this, even today the National and International Guidelines advise against the execution of craniotomy (which is usually performed with the use of an electric suction saw), although it is an important fact from a scientific point of view, as bone dust in these cases it could be highly infectious. To minimize this risk for the operator and eliminate it, to date there are only individual protective devices, i.e. diving suits that cover the head of the operator performing the craniotomy. This method is very expensive and not very applicable, as it provides that each autopsy room has at least three diving suits because a regular autopsy examination is usually carried out by a first operator and room assistants. However, even if these devices are used, they do not reduce the infectious risk, as, during the craniotomy, the bone dust is released into the air of the autopsy room and is deposited everywhere. This eventuality increases the costs of sanitizing the environments. In fact, sanitation is one of the containment measures of coronaviruses (Coronaviridae), that is a large family of microorganisms existing in nature in the form of positive-stranded RNA viruses capable of infecting vertebrates. The family includes the Orthocoronavirinae subfamily to which the human-transmitted beta coronavirus genus belongs. In particular, seven viruses belonging to the beta-coronavirus genus are currently known to cause transmissible respiratory infections in humans, including serious diseases such as Middle East Respiratory Syndrome (MERS) and Severe Acute Respiratory Syndrome (SARS). These also include the new coronavirus, 2019-nCOV (now called SARS-CoV-2) capable of causing a disease known as COVID-19 (COronaVIrus Disease 19). The cases described in the literature show that the virus determines a spectrum of symptoms and clinical signs that are highly variable in terms of incubation, onset and severity. These include dry cough, fever, asthenia, nasal congestion, diarrhea up to a picture of atypical interstitial pneumonia (with ground glass appearance), severe acute respiratory syndrome, acute renal failure, death. The methods of transmission occur from person to person by means of saliva, close contacts or with contaminated hands for which hand hygiene, respect for the safety distance and avoid touching eyes, nose and mouth are recommended. To date, many states are adopting increasingly restrictive measures aimed at limiting interpersonal contacts and therefore the transmission of the virus to a minimum.

### SUMMARY

The object of the present invention is to provide a cranial autopsy protective device for the autopsy of infected corpses which allows to safely perform the craniotomy of the corpses, even infected ones, thus having characteristics such as to overcome the limits of the current known protection devices.

According to the present invention, a cranial autopsy protective device for the autopsy of infected corpses is realized.

### BRIEF DESCRIPTION OF THE FIGURES

For a better understanding of the present invention, a preferred embodiment is now described, purely by way of non-limiting example, with reference to the attached drawings, in which:

FIG. 1 shows a cranial autopsy protective device for the autopsy of infected corpses, according to the invention.

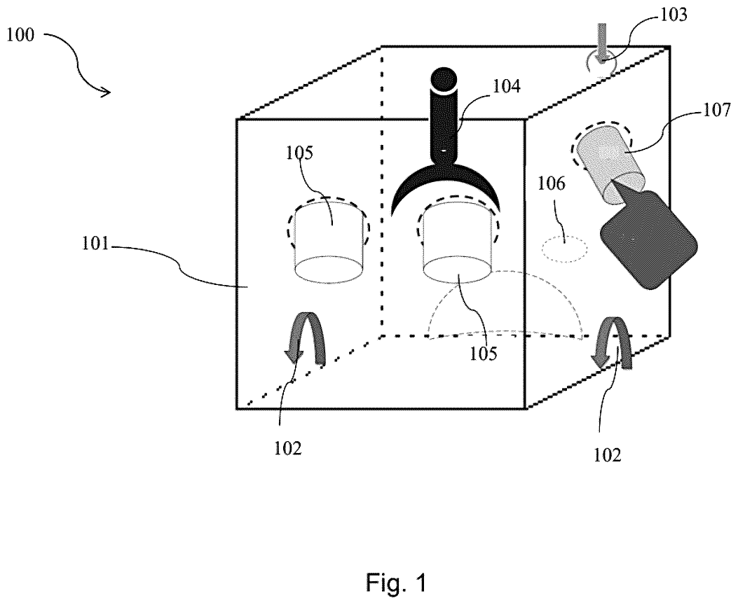

### DETAILED DESCRIPTION

With reference to this FIGURE, a cranial autopsy protective device 100 for the autopsy of infected corpses is shown, comprising an aspirating case 101 configured to be positioned on the head of a corpse before carrying out the autopsy, allowing total isolation of the corpse head.

According to one aspect of the invention, the aspirating case 101 comprises steel hooks 102 that are adjustable and connectable to the autopsy table for the correct adhesion of the case to the autopsy table.

According to another aspect of the invention, the aspirating case 101 comprises a valve 103 with washable rubber insulation through which a nebulization of ozone and chlorine is performed, suitable for sanitizing the case after the craniotomy has been performed.

According to another aspect of the invention, the aspirating case 101 comprises an adjustable steel head fixator 104.

According to another aspect of the invention, the aspirating case 101 comprises first openings 105 comprising a system of insulating anti-cut gloves. The operator, putting his hands inside these openings 105, automatically wears gloves in order to close the case hermetically under negative pressure in a non-infected way.

According to another aspect of the invention, the aspirating case 101 includes a second opening 106 for the insertion of the electric saw with which the operator performs the craniotomy.

According to another aspect of the invention, the aspirating case 101 comprises a third opening 107 comprising an insulating system to accommodate an electric or battery-operated suction unit having a disposable replaceable bag.

According to another aspect of the invention, the aspirating case 101 includes a fourth opening 108 comprising a washable rubber insulation and configured for the insertion of the electric cable of the electric saw.

According to another aspect of the invention, the aspirating case 101 is made of light and anti-breakage material, for example in plexiglass.

Therefore, the cranial autopsy protective device according to the invention allows craniotomies to be made of all infected corpses.

Another advantage of the cranial autopsy protective device according to the invention is that it can be used for all types of infections and pathogens.

Finally, the cranial autopsy protective device according to the invention reduces the management costs of a craniotomy.

Finally, it is clear that modifications and variations may be made to the cranial autopsy protective device for the autopsy of infected corpses described and illustrated here without thereby departing from the protective scope of the present invention, as defined in the attached claims.

1-7. (canceled)

8. A cranial autopsy protective device for the autopsy of infected corpses, comprising: an aspirating case configured to be positioned on the head of a body before the execution of the autopsy allowing the total isolation of the head of the body, the aspirating case comprising adjustable steel hooks connectable to an autopsy table for the correct adhesion of the aspirating case to the autopsy table, wherein the aspirating case comprises a valve with washable rubber insulation through which a nebulization of ozone and chlorine suitable for sanitize the aspirating case is performed after the execution of a craniotomy.

9. The cranial autopsy protective device according to claim 8, wherein the aspirating case comprises an adjustable steel head fixator.

10. he cranial autopsy protective device according to claim 8, wherein the aspirating case comprises first openings comprising a system of insulating cut-resistant gloves.

11. The cranial autopsy protective device according to claim 8, wherein the aspirating case comprises a second opening for the insertion of an electric saw for performing a craniotomy.

12. The cranial autopsy protective device according to claim 8, wherein the aspirating case comprises a third opening comprising an insulating system for accommodating an electric or battery-powered aspirator having a replaceable disposable bag.

13. The cranial autopsy protective device according to claim 8, wherein the aspirating case comprises a fourth opening, comprising a washable rubber insulation and configured for the insertion of the electric cable of an electric saw.

14. The cranial autopsy protective device according to claim 8, wherein the aspirating case is made of light and anti-breaking material.
